(19) United States
(12) Patent Application Publication
(10) Pub. No.: US 2025/0322946 A1
(43) Pub. Date: Oct. 16, 2025

(54) Method for determining an objective fatigue level of an operator performing a mission, associated determination system and method for estimating and determining such a fatigue level

(71) Applicant: THALES, Meudon (FR)

(72) Inventors: Bastien BERTHELOT, MERIGNAC (FR); Vincent IBANEZ, MERIGNAC (FR)

(21) Appl. No.: 19/171,357

(22) Filed: Apr. 7, 2025

(30) Foreign Application Priority Data
Apr. 12, 2024 (FR) 2403805

(51) Int. Cl. G16H 40/63

(52) U.S. Cl. CPC G16H 40/63

(57) ABSTRACT

A method for determining an objective level of fatigue of an operator performing a mission, the method including acquisition of a first estimation of fatigue from a biomathematics model of prediction of fatigue from plurality of mission data related to the mission, acquisition of a second estimation of fatigue from a physiological model of prediction of fatigue from physiological data of the operator, and determination of an objective level of fatigue by merging the two estimations of fatigue.

### CROSS-REFERENCE TO RELATED APPLICATIONS

This application is a U.S. non-provisional application claiming the benefit of French Application No. 24 03805, filed on Apr. 12, 2024, which is incorporated herein by reference in its entirety.

### TECHNICAL FIELD OF THE INVENTION

The present invention relates to a method for determining an objective level of fatigue of an operator performing a mission.

The present invention also relates to a determination system using such a determination method.

The present invention further relates to a method of estimation and of determination such a level of fatigue.

The invention relates more particularly to the technical field of determining the fatigue of an operator.

### BACKGROUND OF THE INVENTION

The operator operates, e.g., in a critical operational context. In other words, the fatigue of the operator in such critical context can lead to significant consequences. Such is particularly the case in the fields of aeronautics, aerospace, railway, nuclear, medical, etc.

In the aeronautical field, biomathematics models for predicting fatigue are known in particular. Such models provide tools for predicting the levels of fatigue of crew members. Predictions are based on scientific knowledge of factors generating fatigue and on mission planning information. Such models are used as a solution for predicting fatigue and are currently available on the market.

However, existing biomathematics fatigue models cannot accurately and objectively estimate levels of fatigue for any type of mission and operator. Indeed, such biomathematics models are generally based on average levels of fatigue and other data coming from ad hoc campaigns such as surveys or reports obtained from a limited number of individuals. However, the surveys and statements are generally based on personal sensations experienced by operators, that may be biased by cultural, professional or operational factors. Same do not correspond to an objective way of estimating a level of fatigue.

Furthermore, existing biomathematics models rest on generic principles and do not always reflect the reality of operations. Same do not take into account operational factors such as weather, season, or the type of transportation used for the mission.

### SUMMARY OF THE INVENTION

The aim of the invention is then to propose a means of objectively and accurately determining the level of fatigue of an operator.

To this end, the subject matter of the invention relates to a method of determining a level of fatigue of an operator performing a mission, the method including:
acquisition of a first estimation of fatigue determined by a biomathematics model of prediction of fatigue from a plurality of mission data related to the mission;
acquisition of a second estimation of fatigue determined by a physiological model of prediction of fatigue from physiological data of the operator; and
determination of an objective level of fatigue by merging the two estimations of fatigue.

According to other advantageous aspects of the invention, the determination method includes one or a plurality of the following features, taken individually or according to all technically possible combinations:
the merging is performed by the fitting of the first estimation of fatigue using the second estimation of fatigue;
the fitting is done using an optimal filter;
the optimal filter is a Kalman filter and/or a particulate filter;
the implementation of the optimal filter includes:
   calculation of an initial weight for a time T from the first estimation of fatigue acquired for a preceding time T-1, the initial weight corresponding to the uncertainty of the first estimation of fatigue;
   prediction of a first theoretical estimation of fatigue for time T from the initial weighting for time T, the first estimation of fatigue acquired for the preceding time T-1 and a function of theoretical evolution of fatigue;
   modification of the initial weight from a conditional probability of the second estimation of fatigue acquired for time T knowing the first estimation of theoretical fatigue for time T; and
   estimation of an objective level of fatigue for time T from the initial weighting for modified time T and the first estimation of theoretical fatigue for time T;
the implementation of the optimal filter further includes a generation a set of particles, each particle including a first estimation of fatigue acquired for a preceding time T-1 and a priorly undefined initial weighting associated with the first estimation of fatigue;
the calculation, prediction, modification and estimation being implemented for each of the particles;
the method further includes estimation of an objective level of fatigue resulting from a weighted average of the estimations of the level of fatigue for the different particles.
   the merging is performed by a supervised learning algorithm apt to extract a supervised set of signature(s) related to the objective level of fatigue from the two estimations of fatigue, a model of the supervised learning algorithm being chosen from the group consisting of: neural networks, logistic regression, support vector machine, and k-nearest neighbor algorithm;
   the merging is performed by an unsupervised learning algorithm apt to extract an unsupervised set of signature(s) related to the objective level of fatigue from the two estimations of fatigue, a model of the unsupervised learning algorithm being chosen from the group consisting of: hierarchical grouping, partitioning into K-means, self-organizing maps, and Gaussian mixing;
   the biomathematics model is constructed from subjective and/or statistical data relating to a plurality of individuals.
   the operator's mission is the piloting of an aircraft and wherein the plurality of mission data include at least one type of data selected from the group including:
      data relating to the schedule of a crew to which the operator belongs;
      data relating to the configuration of an airline.
   physiological data include at least one type of data selected from the group consisting of:
      images of the operator;
      a heart rate;
      blood pressure;
      oxygen respiration;
      sweating;
      oxygen saturation;
      a level of dehydration;
   the physiological data of the operator are measured during the mission.

The invention further relates to a system for determining an objective level of fatigue including technical means configured to implement the method according to any of the features described hereinabove.

The invention further relates to a method of determination of a level of fatigue of an operator, including:
determination of a first estimation of fatigue determined by a biomathematics model of prediction of fatigue from a plurality of mission data related to the mission;
determination of a second estimation of fatigue determined by a physiological model of prediction of fatigue from physiological data of the operator; and
implementation of the determination method according to any of the features described hereinabove.

### BRIEF DESCRIPTION OF THE DRAWINGS

The invention will be clearer upon reading the following description, given only as an example, but not limited to, and making reference to the drawings wherein:

FIG. 1 is a schematic representation of an architecture of estimation and determination of level of fatigue of an operator, according to the invention;

FIG. 2 is a schematic representation of system for determining an objective level of fatigue, the determination system being part of the estimation and determination architecture shown in FIG. 1; and

FIG. 3 is an organization chart of a method of estimation and determination of a level of fatigue, the method being implemented by the architecture shown in FIG. 1.

### DETAILED DESCRIPTION

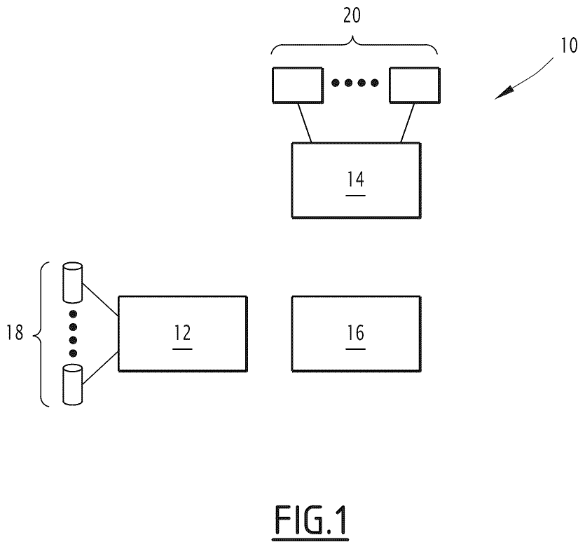

FIG. 1 shows an architecture 10 of estimation and determination of an operator's level of fatigue. The architecture 10 makes it possible to estimate and determine an operator's level of fatigue.

Advantageously, the architecture 10 may be used in the aeronautical field. In such a case, the operator is part of the flight crew, in particular of the commercial flight crew. In other examples, the operator is one of the flight planning operators or of the maintenance operators or of the aircraft control operators or of the air traffic controllers.

Advantageously, the operator is a pilot apt to pilot an aircraft.

The term “aircraft” refers to any flying craft that may be piloted from the cockpit of the aircraft, as is the case, e.g., with an airplane or helicopter, or else at a distance therefrom, as is the case, e.g., of a drone.

In general, the notion of operator may apply to any other person performing a critical mission, e.g., in the field of transport (rail or heavy goods vehicles or, e.g., any transport) or in the nuclear or space field, or in medicine.

As indicated hereinabove, the operator performs a mission that is determined by the field of their activity.

More particularly, the mission of the operator includes a plurality of tasks defined according to the skills of the operator.

When the operator is an aircraft pilot, their mission is generally to fly the aircraft from a point of departure to a point of destination.

The estimation and determination architecture 10 includes a first system of estimation of fatigue 12, a second system of estimation of fatigue 14 and a system of objective determination of fatigue 16.

The first fatigue estimation system 12 is configured to determine a first estimation of fatigue from mission data, implementing a biomathematics model of prediction of fatigue.

To this end, the first fatigue estimation system 12 is connected to one or a plurality of databases 18 containing mission data. The system 12 may, e.g., be connected to the database or the databases 18, either directly or indirectly, via, e.g., a computer network.

The mission data describe the mission to be performed by the operator.

These mission data include, e.g., the mission hours (i.e. start, end, duration), the position to be occupied by the operator during the mission (pilot, co-pilot or other critical position, e.g., in the nuclear field or in air traffic control), the composition of the team (e.g., the number of pilots needed for the flight to be carried out), the position of the working day in the period (i.e. the day in the work sequence), information on the mission scheduling (scheduled, rescheduled mission, and in the latter case, anteriority of the rescheduling), time range of the mission (morning, during the day, during the evening, night), type of aircraft and any other work station on which the mission would take place.

Advantageously, when the operator's mission is the piloting of an aircraft, the mission data 18 include at least one type of data chosen from the group including:
data relating to the schedule of a crew to which the operator belongs;
data relating to the configuration of an airline.

Advantageously, the mission data comes from the airline for which the operator performs their mission. In such a case, the database or databases 18 are advantageously made available to the first estimation system 12 by the airline.

The biomathematics model of prediction of fatigue is, e.g., a model known per se which serves to determine a level of fatigue from the mission data, as defined hereinabove. The biomathematics model is, e.g., constructed from subjective and/or statistical data relating to a plurality of individuals. Each of the individuals [re] presents, e.g., an operator performing an identical or similar mission to the mission of the operator for whom the level of fatigue is determined by the estimation and determination architecture 10.

Advantageously, the data used to construct the biomathematics model are determined beforehand from subjective and/or statistical data relating to said individuals. The data are collected, e.g., following self-statements by the individuals and/or spot survey campaigns. The data may also be treated statistically to make same applicable to other individuals.

For example, a biomathematics model may be constructed or adjusted from declarative data from pilots following each flight. The pilot may state, e.g., their subjective level of fatigue after each flight. The model may thereby associate different flight times with different levels of fatigue experienced by pilots. Thereby, such a model may determine a level of fatigue felt for a given instant during the flight.

A simple biomathematics model may take, e.g., the form of charts associating one value with another. A more complex biomathematics model may take the form of a formula associating a value with a plurality of other values and including coefficients determined beforehand from subjective and/or statistical data relating to individuals.

The level of fatigue determined by the biomathematics model has a value (e.g., a number or a character) determined on a scale specific to the model.

The second fatigue estimation system 14 is configured to determine a second estimation of fatigue from the physiological data of the operator, implementing a physiological model of prediction of fatigue.

The physiological data of the operator present any type of data for characterizing the physical state of the operator. The physiological data are advantageously acquired during the mission or just before the mission or else just after the mission.

Advantageously, the physiological data of the operator include at least one type of data chosen from the group including:
images of the operator;
a heart rate;
a blood pressure;
oxygen respiration;
a respiration rate;
an amplitude of respiration;
sweating;
an oxygen saturation;
a level of dehydration.

In order to acquire physiological data, the second estimation system 14 is connected directly or indirectly to a plurality of sensors 20 arranged, e.g., in the operator's workstation. For example, the sensors 20 are arranged in a fixed and/or removable manner in the cockpit of the aircraft piloted by the operator. The sensors 20 may, e.g., be connected to the second estimation system 14 via a computer network.

More particularly, the plurality of sensors 20 includes any sensor serving to acquire the physiological data of the operator.

For example, the plurality of sensors 20 includes a camera (not shown) configured to acquire images of the operator and a heart rate sensor (not shown) for measuring the heart rate of the operator.

The camera is, e.g., oriented toward the operator or has means for orienting same according to the position of the operator.

For example, the operator's heart rate sensor is configured to be positioned around an operator's wrist.

To this end, the heart rate sensor has, e.g., a bracelet that may be attached to the wrist of the operator and a sensitive part which is intended to measure the heart rate of the operator when the bracelet is attached to their wrist.

The measurement of the heart rate is done, e.g., by the sensitive part, by the technique called photoplethysmography, called PPG. Alternatively, the sensitive part is configured to carry out the measurement of the heart rate on the basis of an analysis of the electrical response between the wrist of the operator, or by analysis of radar signals propagating through the wrist of the operator.

In certain examples, the heart rate sensor is configured to measure other physiological parameters of the operator, such as the blood pressure, oxygen respiration, sweating, level of dehydration, etc.

For oxygen saturation, the heart rate sensor is configured, e.g., to transmit, toward the skin of the operator, and to receive, a light signal including at least two wavelengths. A first wavelength corresponding to a wavelength absorbed by saturated red blood cells, and a second wavelength corresponding to a wavelength absorbed by unsaturated red blood cells. To determine the oxygen saturation, the heart rate sensor is then configured to compare the light intensity received in response to each of the two wavelengths.

In general, the heart rate sensor may be in the form of a connected watch to measure, e.g., their heart rate.

Of course, the aforesaid functions of the heart rate sensor may form separate sensors.

The physiological fatigue prediction model is configured to determine a level of fatigue of an operator using their physiological data as described hereinabove. The level of fatigue has a value (e.g., a number or a character) determined on a scale specific to the model.

To determine the level of fatigue, the physiological model analyzes the physiological data according to predetermined algorithms and associates a level of fatigue according to the analysis.

For example, the physiological model may analyze the operator's images in order to determine the number of eye blinks, the frequency of yawning, and/or any other movement that may be caused by fatigue. The physiological model may analyze, e.g., the operator's heart rate and couple same with information about their movements.

Each of the estimation systems 12, 14 described hereinabove has, e.g., at least partially a hardware component (such as a programmable logic circuit such as an FPGA) and/or a software component. In the latter case, the software component is, e.g., implemented by a computer including at least one processor and a random-access memory. The corresponding model is stored, e.g., in a non-volatile memory of the computer.

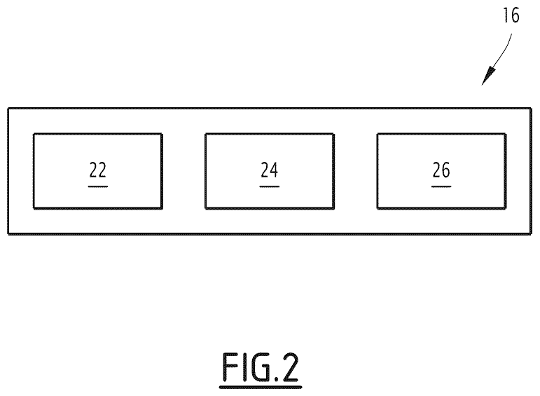

FIG. 2 illustrates in detail the system of objective determination of fatigue 16. The system 16 is configured to merging the first and second estimations of fatigue in order to determine an objective level of fatigue. For this purpose, the system of objective determination of fatigue 16 includes an acquisition module 22, a processing module 24 and an output module 26.

The acquisition module 22 is configured to acquire data coming from the first and second fatigue estimation systems 12, 14. In other words, the acquisition module 22 is configured to acquire the first estimation of fatigue from the first fatigue estimation system 12 and the second estimation of fatigue from the second fatigue estimation system 14.

The processing module 24 is connected to the output of the acquisition module 22 and is configured to determine an objective level of fatigue by merging the first estimation of fatigue and the second estimation of fatigue, as will be explained in more detail thereafter.

As an optional addition, the processing module 24 is further apt to identify a set of risk factors associated with the objective level of fatigue.

The output module 26 is connected to the output of the processing module 24. The output module 26 is apt to transmit the objective level of fatigue to a system external to the architecture 10.

Each of the modules 22, 24, 26 of the system of objective determination of fatigue 16 described above has, e.g., at least partially a hardware component (such as a programmable logic circuit such as an FPGA) and/or a software component. In the latter case, the software component is, e.g., implemented by a computer including at least one processor and a random-access memory.

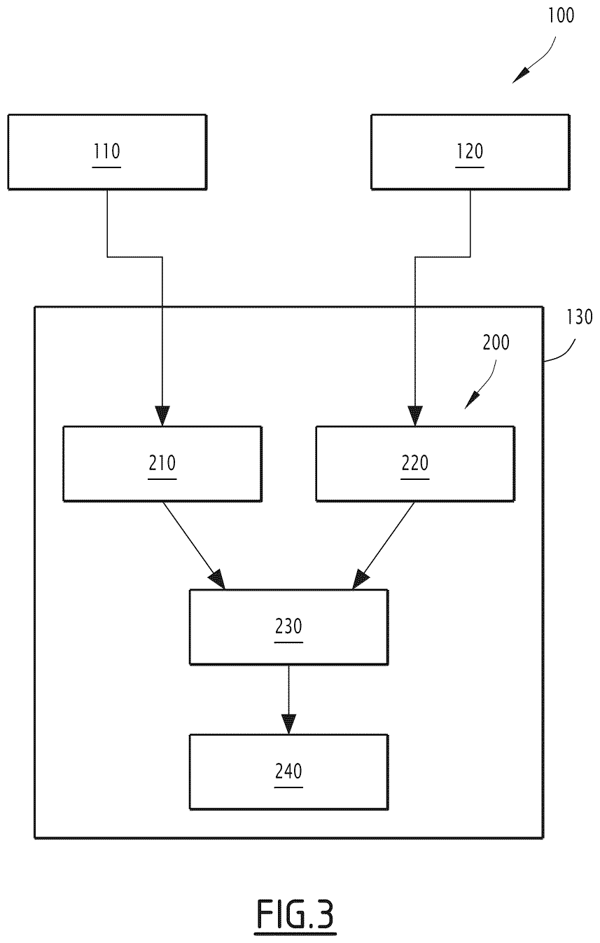

The operation of the estimation and determination architecture 10 according to the invention will now be described with reference to FIG. 3 representing a flowchart of the method 100 according to the invention, of estimation and determination of a level of fatigue of the operator. The method is implemented by the architecture 10.

During operation 110 of the estimation and determination method 100, the first fatigue estimation system 12 determines a first estimation corresponding to the level of fatigue determined by the biomathematics model, as described hereinabove.

Such level of fatigue is determined using mission data related to the operator's mission.

The operation 110 is, e.g., implemented before the operator's mission. In certain embodiments, the operation 110 may also be implemented during the mission, e.g., throughout the mission. In such a case, the first estimation may present a series of data generated at different times by the biomathematics model.

During operation 120 of the estimation and determination method 100, the second fatigue estimation system 14 determines a second estimation corresponding to the level of fatigue determined by the physiological model, as described hereinabove. Operation 120 may be implemented following operation 110 or in parallel with the operation 120.

The operation 120 is, e.g., implemented during the operator's mission by using physiological data also acquired during the mission. For example, the operation is implemented continuously throughout the operator's mission. Thereby, the second estimation may present a series of data generated at different times by the physiological model.

During operation 130 of the estimation and determination method 100, the system of objective determination of fatigue 16 implements a method 200 of determination of the objective level of fatigue. The method 200 is based on the two estimations determined during operations 110 and 120. During operation 210 of the determination method 200, the system of objective determination of fatigue 16 acquires, via the acquisition module 22 thereof, the first estimation of fatigue determined during operation 110.

During operation 220, which may be implemented following operation 210 or in parallel with operation 220, the acquisition module 22 acquires the second estimation determined during operation 120.

During the next operation 230, the processing module 24 processes the estimations received in order to determine an objective level of fatigue.

To this end, according to certain embodiments, the processing module 24 may first normalize the estimations received. Thereof may be the case, e.g., when the biomathematics and physiological models do not use the same determination scale for the fatigue.

Then, the processing module 24 merges the estimations received.

The merging is performed, e.g., by the fitting of the first estimation of fatigue using the second estimation of fatigue.

Preferably, the fitting is done using an optimal filter such as a Kalman filter or a particle filter.

According to one example of embodiment, the optimal filter is a Kalman filter.

The Kalman filter first calculates an initial weight for a time T from the first estimation of fatigue acquired for a preceding time T-1. The initial weighting corresponds to the uncertainty of the first estimation of fatigue.

The Kalman filter then predicts a first theoretical estimation of fatigue for time T from the initial weighting for time T, the first estimation of fatigue acquired for the preceding time T-1 and a function of theoretical evolution of fatigue.

The theoretical evolution of fatigue function is used to estimate the evolution of the first estimation of fatigue over time. For example, the function of theoretical evolution of fatigue corresponds to a piecewise linear function according to the phases of sleep or of awakening of the operator.

In a variant, the theoretical evolution of fatigue function of the Kalman filter corresponds to a more complex function depending on the data relating to the schedule of a crew to which the operator belongs, such as the duration of the mission, or else the time of the mission carried out during the night.

The theoretical evolution model of fatigue of the Kalman filter fatigue is modeled, e.g., according to the following equation:

$${x}_{bio , T} = f ( {x}_{bio , T - 1} ) + {w}_{T}$$

where
x_{bio,T-1} corresponds to the first estimation of fatigue at the preceding time T-1;
x_{bio,T} corresponds to the first theoretical estimation of fatigue at time T;
ƒ corresponds to the theoretical evolution of fatigue function; and
w^{T} corresponds to the initial weighting at time T.

The Kalman filter then modifies the initial weighting from a conditional probability of the second estimation of fatigue acquired for the preceding time T-1 knowing the first estimation of theoretical fatigue for the preceding time T-1.

Finally, the Kalman filter estimates an objective level of fatigue for time T from the initial weighting for the modified time T and the first theoretical estimation of fatigue for time T.

Alternatively, the optimal filter is a particulate filter.

The method with the particulate filter first generates a set of particles. Each particle includes a first estimation of fatigue acquired for a preceding time T-1 and a priorly undefined initial weighting associated with the first estimation of fatigue.

The method with the particulate filter has operations that are in part similar to the Kalman filter. Thereby, the method with the particle filter implements the operations of calculation, prediction and modification of the Kalman filter as described hereinabove.

Following the modification, the method with the particle filter performs, e.g., the operation of resizing the set of particles while keeping certain particles according to the modified initial weightings thereof. Resizing is used, e.g., for forming a modified set of particles with fewer particles than the set of particles and where the particles with the highest weightings are more likely to be chosen.

The calculation, prediction, modification and estimation operations are implemented for each particle.

In such a case, the implementation of the optimal filter further includes a sub-operation of estimation of an objective level of fatigue resulting from a weighted average of the estimations of the level of fatigue for the different particles. It is thus the resulting objective level of fatigue which is subsequently considered as the objective level of fatigue determined by the determination method 200.

In a variant, the merging is carried out by automatic learning on the data used by the biomathematics model 12 applied to new cases of the fatigue prediction system 14.

Preferably, the merging is performed by a supervised learning algorithm apt to extract a supervised set of signature(s) related to the objective level of fatigue from the two estimations of fatigue, a model of the supervised learning algorithm being chosen from the group consisting of: neural networks, logistic regression, support vector machine, and k-nearest neighbor.

For example, the supervised learning method is symbolic learning based on initial knowledge of the biomathematics model. The initial knowledge is constantly developing as new rules may be added to initial knowledge while maintaining consistency with the initial knowledge and generalizing knowledge learned. For example, the type of symbolic learning is learning by detection of similarities from examples and counter-examples. For example, the type of symbolic learning is learning by seeking explanations from examples and counter-examples.

According to another example, the learning method is digital learning. For example, the type of digital learning is performed via one or a plurality of statistical models.

In a variant, the merging is performed by an unsupervised learning algorithm apt to extract an unsupervised set of signature(s) related to the objective level of fatigue from the two estimations of fatigue, a model of the unsupervised learning algorithm being chosen from the group consisting of: hierarchical grouping, partitioning into K-means, self-organizing maps, and Gaussian.

According to certain examples, the determination method 200 further includes an optional operation during which the processing module 24 identifies a set of risk factors associated with the objective level of fatigue.

During operation 240 of the determination method 200, the processing module 24 transmits to the output module 26 the determined objective level of fatigue, accompanied, if appropriate, by the associated risk factors.

The output module 26 then transmits the data to any interested system. Such a system may, e.g., include a means of display, a database and/or a remote server. In certain examples, the objective level of fatigue and, if appropriate, the risk factors may also be communicated to the operator.

In this way, it may be understood that the present invention has a certain number of advantages. More particularly, the invention serves to objectively and precisely determine the objective level of fatigue. Thereby, independently of the database or databases used to determine a first estimation of fatigue and independently of the sensors 20 used to determine a second estimation of fatigue, the invention serves to determine an objective level of fatigue which is standardized and thus comparable among a plurality of operators and which may be used to subsequently identify the set of risk factors associated with the objective level of fatigue.

Supervised learning further improves the accuracy of the objective level of fatigue due to a considerable experience merging the two estimations.

Unsupervised learning improves the calculation speed merging the two estimations.

Furthermore, the use of an optimal filter makes it possible to have control over the merging of estimations. When the optimal filter is a particulate filter, the determined objective level of fatigue is particularly accurate. The Kalman filter makes it possible to obtain a good approximation faster by simplifying the calculations.

1. A method of determining an objective level of fatigue of an operator performing a mission, the method comprising:
acquiring a first estimation of fatigue from a biomathematics model of prediction of fatigue from a plurality of mission data related to the mission;
acquiring a second estimation of fatigue from a physiological model of prediction of fatigue from physiological data of the operator; and
determining an objective level of fatigue comprising merging the two estimations of fatigue.

2. The method according to claim 1, wherein said merging comprises fitting the first estimation of fatigue using the second estimation of fatigue.

3. The method according to claim 2, wherein said fitting comprises implementing an optimal filter.

4. The method according to claim 3, wherein the optimal filter is a Kalman filter.

5. The method according to claim 4, wherein said implementing comprises:
calculating an initial weight for a time T from the first estimation of fatigue acquired for a preceding time T-1, the initial weight corresponding to an uncertainty of the first estimation of fatigue;
predicting a first theoretical estimation of fatigue for time T from the initial weighting for time T, the first estimation of fatigue acquired for the preceding time T-1 and a theoretical evolution of fatigue function;
modifying the initial weight from a conditional probability of the second estimation of fatigue acquired for time T knowing the first estimation of theoretical fatigue for time T; and
estimating an objective level of fatigue for time T from the initial weighting for time T modified and the first estimation of theoretical fatigue for time T.

6. The method according to claim 5, wherein said implementing comprises generating a set of particles, each particle comprising a first estimation of fatigue acquired for a preceding time T-1 and a priorly undefined initial weighting associated with the first estimation of fatigue, said calculating, predicting, modifying and estimating being implemented for each particle, and wherein said implementing further comprises estimating an objective level of fatigue resulting from a weighted average of the estimations of the level of fatigue for the different particles.

7. The method according to claim 1, wherein said merging is performed by a supervised learning algorithm that extracts a supervised set of signature(s) related to the objective level of fatigue from the two estimations of fatigue, a model of the supervised learning algorithm being chosen from the group consisting of: neural networks, logistic regression, support vector machine, and k-nearest neighbor.

8. The method according to claim 1, wherein said merging is performed by an unsupervised learning algorithm that extracts an unsupervised set of signature(s) related to the objective level of fatigue from the two estimations of fatigue, a model of the unsupervised learning algorithm being chosen from the group consisting of: hierarchical grouping, partitioning into K-means, self-organizing maps, and Gaussian mixing.

9. The method according to claim 1, wherein the biomathematics model is constructed from subjective and/or statistical data relating to a plurality of individuals.

10. The method according to claim 1, wherein the operator's mission is piloting of an aircraft, and wherein the plurality of mission data comprise at least one type of data selected from:
data relating to the schedule of a crew of which the operator belongs, and
data relating to the configuration of an airline.

11. The method of claim 1, wherein the physiological data comprises at least one type of data selected from the group consisting of:
images of the operator;
a heart rate;
a blood pressure;
oxygen respiration;
sweating;
oxygen saturation; and
a level of dehydration.

12. The method according to claim 1, wherein the physiological data of the operator are measured during the mission.

13. A system for determining an objective level of fatigue comprising technical means configured to carry out the method according to any claim 1.

14. A method for estimating and determining a level of fatigue of an operator, comprising:
determining a first estimation of fatigue from a biomathematics model of prediction of fatigue from plurality of mission data related to the mission;
determining of a second estimation of fatigue from a physiological model of prediction of fatigue from physiological data of the operator; and
determining an objective level of fatigue comprising merging the two estimations of fatigue.
